(19) United States
(12) Patent Application Publication
(10) Pub. No.: US 2024/0404045 A1
(43) Pub. Date: Dec. 5, 2024

(54) A Monitoring And Evaluation Method For Comprehensive Evaluation Index Of Machine-Harvested Cotton Defoliation Effect And System Thereof

(71) Applicants: Shihezi University, Shihezi, Xinjiang (CN); Agricultural Technology Extension Station of the Fourth Division of Xinjiang Production, Kekedala, Xinjiang (CN)

(72) Inventors: Ze Zhang, Shihezi, Xinjiang (CN); Yiru Ma, Shihezi, Xinjiang (CN); Aiqun Chen, Shihezi, Xinjiang (CN); Xin Lv, Shihezi, Xinjiang (CN); Tongyu Hou, Shihezi, Xinjiang (CN); Xiangyu Chen, Shihezi, Xinjiang (CN); Lulu Ma, Shihezi, Xinjiang (CN); Qiang Zhang, Shihezi, Xinjiang (CN)

(21) Appl. No.: 18/030,888

(22) PCT Filed: Aug. 23, 2022
(86) PCT No.: PCT/CN2022/114077

§ 371 Date: Aug. 6, 2024

(30) Foreign Application Priority Data
Apr. 15, 2022 (CN) 202210396435.7

(51) Int. Cl. G06T 7/00; G01N 21/27; G01N 33/00; G06T 7/40; G06T 7/90

(52) U.S. Cl. CPC G06T 7/0012; G01N 21/27; G01N 33/0098; G06T 7/40; G06T 7/90; G06T 2207/10024; G06T 2207/20021; G06T 2207/20081; G06T 2207/30188

(57) ABSTRACT

A monitoring and evaluation method for comprehensive evaluation index of machine-harvested cotton defoliation effect and system thereof relates to the field of monitoring and evaluation of cotton-harvested indexes. The RGB images of the machine-harvested cotton canopy are firstly acquired to extract visible-light vegetation index features, color component features and texture features of machine-harvested cotton canopy RGB images; the visible-light vegetation index features, color component features and texture features are input to the trained comprehensive evaluation model of machine-harvested cotton defoliation effect, to output the defoliation effect evaluation value; finally, the harvesting timing of machine-harvested cotton is determined according to the defoliation effect evaluation value. The invention can improve the accuracy and efficiency of monitoring and evaluating the comprehensive evaluation index of the machine-harvested cotton defoliation effect and provide a reference for the machine-harvested cotton defoliation effect research and the machine-harvested cotton best harvesting time determination.

### 1. TECHNICAL FIELD

The invention relates to the field of monitoring and evaluation of cotton harvesting index, in particular to a monitoring and evaluation method for comprehensive evaluation index of machine-harvested cotton defoliation effect and system thereof.

### 2. BACKGROUND ART

Cotton is one of China's main cash crops and the most critical fiber crop for the textile industry. Cotton production plays an essential role in international trade and national security and is also an important economic source for cotton farmers. With the increasing acreage of machine-harvested cotton, there has been a gradual increase in research on the effect of cotton defoliation. In agricultural production, the defoliation rate and the boll-opening rate are regarded as the basis for cotton harvesting, where it is possible to carry out mechanical harvesting with the machine-harvested cotton defoliation rate of 90% or more and boll-opening rate of 95% or more. Therefore, it is essential to monitor and evaluate the index of defoliation rate, spat rate and yield of machine-harvested cotton, for research related to defoliants in machine-harvested cotton and to determine the harvesting time of machine-harvested cotton in field production.

However, the existing monitoring and evaluation methods for the comprehensive evaluation of machine-harvested cotton defoliation index are usually more subjective, subject to the influence of human factors, which results in the low accuracy in the evaluation of machine-harvested cotton defoliation effect and the harvesting time determination, and long and inefficient cycle. Therefore, it is an urgent problem to be solved for the improvement of the accuracy and efficiency for monitoring and evaluating the comprehensive evaluation index of machine-harvested cotton defoliation effect.

### 3. SUMMARY OF THE INVENTION

The purpose of the invention is to provide a monitoring and evaluation method for comprehensive evaluation index of machine-harvested cotton defoliation effect and system thereof, to improve the accuracy and efficiency of monitoring and evaluating the comprehensive evaluation index of machine-harvested cotton defoliation effect, and to provide reference for studying the machine-harvested cotton defoliation effect and determining the optimal harvesting time of machine-harvested cotton.

To achieve the above purpose, the invention provides the following technical solutions:

On the one hand, the invention provides a monitoring and evaluation method for comprehensive evaluation index of machine-harvested cotton defoliation effect, comprising the following steps:
acquiring RGB images of machine-harvested cotton canopy;
extracting the visible-light vegetation index features, color component features and texture features of the machine-harvested cotton canopy RGB images;
inputting the visible-light vegetation index features, color component features and texture features into a trained machine-harvested cotton defoliation effect comprehensive evaluation model to output defoliation effect evaluation values; the trained comprehensive evaluation model of machine-harvested cotton defoliation effect is an extreme learning machine model based on particle swarm optimization algorithm through training with visible-light vegetation index features, color component features and texture features of the machine-harvested cotton canopy RGB images as input and the defoliation effect evaluation value as output; the extreme learning machine model comprising an input layer, a hidden layer and an output layer, and the particle swarm optimization algorithm provided for optimizing the weight values of the input layer and the bias values of the hidden layer;
determining the harvesting timing of machine-harvested cotton based on the defoliation effect evaluation value.

Optionally, the method further comprises the following step after acquiring the machine-harvested cotton canopy RGB image:
stitching the machine-harvested cotton canopy RGB image by Pix4Dmapper software to obtain a machine-harvested cotton canopy RGB ortho-image.

Optionally, the step of extracting the visible-light vegetation index features, color component features and texture features of the machine-harvested cotton canopy RGB images, specifically comprises:
dividing the RGB ortho-images of the of machine-harvested cotton canopy according to the location of each test plot in the area to be monitored, to obtain a plurality of regions of interest;
obtaining the digital number of each color channel in each region of interest, and calculating the average digital quantization value of each color channel; the color channels comprising R channel, G channel and B channel;
performing normalization of the digital quantization value and the average digital quantization value of each color channel, and calculating each color component value; the color component value referring to the normalized value of each color component in the RGB ortho-image, and the RGB ortho-image each color component comprising the r component, the g component and the b component;
calculating the visible-light vegetation index features based on the respective color component values;
performing the color space model transformation on the RGB color space model corresponding to the color features in each region of interest, respectively, to obtain a transformed color space model; the transformed color space model comprising an HSV color space model, a La*b* color space model, a YCrCb color space model, and a YIQ color space model;
extracting color component features in each color space model according to the transformed color space model, and calculating the digital number of each the color component feature;
calculating the texture features of angular second moment, entropy, contrast and correlation based on the gray-level co-occurrence matrix.

Optionally, the method further comprises the following step after extracting the visible-light vegetation index features, color component features and texture features of the machine-harvested cotton canopy RGB images:
selecting the extracted visible-light vegetation index features, color component features and texture features using random forest method respectively to obtain the selected image features; the selected image features comprising at least one visible-light vegetation index feature, at least one color component feature, and at least one texture feature.

Optionally, the method further comprises the following steps before acquiring RGB images of machine-harvested cotton canopy:
collecting historical base data of machine-harvested cotton in the area to be monitored;
using principal component analysis to determine a comprehensive evaluation index of the machine-harvested cotton defoliation effect according to the historical base data;
the comprehensive evaluation index of the machine-harvested cotton defoliation effect referring to the index for evaluating the harvesting timing of machine-harvested cotton;
calculating the standard threshold value of machine-harvested cotton defoliation effect comprehensive evaluation according to the machine-harvested cotton defoliation effect comprehensive evaluation index; the standard threshold value of machine-harvested cotton defoliation effect comprehensive evaluation referring to the standard threshold value for evaluating the harvesting timing of machine-harvested cotton; determine the suitability of the machine-harvested cotton for harvesting corresponding to the machine-harvested cotton canopy RGB image.

Optionally, the step of using principal component analysis to determine a comprehensive evaluation index of machine-harvested cotton defoliation effect according to the historical base data, specifically comprises:
performing the normalization of the historical base data to obtain a data matrix corresponding to the historical base data t;
calculating the correlation matrix or the covariance matrix corresponding to the data matrix based on the data matrix;
determining the eigenvalues of the correlation matrix or covariance matrix and calculating the eigenvectors corresponding to each eigenvalue;
determining the principal component eigenvectors based on the eigenvectors and calculating the contribution and cumulative contribution of the principal component eigenvectors;
determining the comprehensive evaluation index of machine-harvested cotton defoliation effect based on the contribution rate and cumulative contribution rate of the principal component eigenvector.

Optionally, the step of calculating the standard threshold value of machine-harvested cotton defoliation effect comprehensive evaluation according to the machine-harvested cotton defoliation effect comprehensive evaluation index, specifically comprises:
according to the comprehensive evaluation index of machine-harvested cotton defoliation effect, the comprehensive evaluation standard threshold of machine-harvested cotton defoliation effect is calculated by the following formula:

PCA1=0.9992×T+0.0008×C
wherein, PCA1 indicates the standard threshold value of machine-harvested cotton defoliation effect comprehensive evaluation, T indicates defoliation rate, and C indicates yield.

Optionally, the step of determining the harvesting timing of machine-harvested cotton based on the defoliation effect evaluation value, specifically comprises:
comparing the size of the defoliation effect evaluation value with the standard threshold value of machine-harvested cotton defoliation effect comprehensive evaluation, and determining the suitability of the machine-harvested cotton for harvesting corresponding to the machine-harvested cotton canopy RGB image based on the comparison result, with the following steps:
determining that the machine-harvested cotton corresponding to the machine-harvested cotton canopy RGB image is suitable for harvesting, when the defoliation effect evaluation value is greater than the threshold value of the machine-harvested cotton defoliation effect comprehensive evaluation;
determining that the machine-harvested cotton corresponding to the machine-harvested cotton canopy RGB image is not suitable for harvesting, when the defoliation effect evaluation value is less than or equal to the threshold value of the machine-harvested cotton defoliation effect comprehensive evaluation.

Optionally, the comprehensive evaluation index of the machine-harvested defoliation effect comprises defoliation rate, boll-opening rate and yield.

On the other hands, the invention also provides a monitoring and evaluation system for comprehensive evaluation index of machine-harvested cotton defoliation effect, comprising:
a machine-harvested cotton canopy RGB image acquisition module for acquiring machine-harvested cotton canopy RGB images;
an image feature extraction module for extracting visible-light vegetation index features, color component features and texture features of the machine-harvested cotton canopy RGB images;
a comprehensive evaluation model module for inputting the visible-light vegetation index features, color component features and texture features into a trained comprehensive evaluation model of machine-harvested cotton defoliation effect to output the defoliation effect evaluation values; the trained comprehensive evaluation model of machine-harvested cotton defoliation effect is an extreme learning machine model based on particle swarm optimization algorithm through training with visible-light vegetation index features, color component features and texture features of the machine-harvested cotton canopy RGB images as input and the defoliation effect evaluation value as output;
a machine-harvested cotton harvesting timing determination module for determining the harvesting timing of machine-harvested cotton based on the defoliation effect evaluation value.

According to the specific embodiments provided by the invention, the invention discloses the following technical effects:

A monitoring and evaluation method for comprehensive evaluation index of machine-harvested cotton defoliation effect and system thereof, related to the field of monitoring and evaluation of cotton-harvested indexes. The RGB images of machine-harvested cotton canopy are firstly acquired to extract visible-light vegetation index features, color component features and texture features of RGB images of machine-harvested cotton canopy; the visible-light vegetation index features, color component features and texture features are input to the trained comprehensive evaluation model of machine-harvested cotton defoliation effect, to output the defoliation effect evaluation value.

The invention can evaluate the machine-harvested cotton defoliation effect according to the defoliation effect evaluation value, and the machine-harvested cotton defoliation effect represents the harvesting time of machine-harvested cotton, which directly determines the suitability of machine-harvested cotton for harvesting. Therefore, it is possible to directly determine the suitability of machine-harvested cotton for harvesting corresponding to the currently taken RGB image of the machine-harvested cotton canopy based on the defoliation effect evaluation value.

The invention adopts an extreme learning machine model based on particle swarm optimization algorithm as a comprehensive evaluation model of machine-harvested cotton defoliation effect, and takes the visible-light vegetation index features, color component features and texture features of machine-harvested cotton canopy RGB images as model inputs, to truly and effectively reflect the machine harvested cotton defoliation effects. Therefore, the invention can accurately and reliably determine the best harvesting time of machine-harvested cotton, improve the accuracy of monitoring and evaluation of the comprehensive evaluation index of machine-harvested cotton defoliation effect, solve the problem of strong subjectivity and low accuracy of the traditional method, to provide estimation technical support for the research related to the machine-harvested cotton defoliation effect, and to provide reference for determining the best harvesting time of machine-harvested cotton in agricultural production.

Moreover, after taking RGB images of machine-harvested cotton canopy and extracting image features, the invention can output defoliation effect evaluation values by inputting the extracted features into the model, to determine the suitability of the current machine-harvested cotton for harvesting according to the values. The invention can improve the monitoring and evaluation efficiency of the machine-harvested cotton comprehensive evaluation index by providing a simpler and faster application, thus solving the problem of long evaluation period and low efficiency of the traditional method.

### 4. BRIEF DESCRIPTION OF ACCOMPANY DRAWINGS

To make the technical solutions provided by the invention more comprehensible, a further description of the invention is given below in combination with the attached drawings and embodiments, and the embodiments are exemplary and not the limitations of the scope of the disclosure. Apparently, the described drawings are merely some embodiments of the application rather than all the embodiments of the application. It should be understood that the application is not limited to the drawings described herein. Based on the drawings in the invention, all other drawings obtained by those of ordinary skill in the art without making creative labor fall within the scope of protection of the invention. The following drawings are not intentionally drawn to actual size and scale but are intended to illustrate the main idea of the invention.

FIG. 1 is a flow chart of a monitoring and evaluation method for comprehensive evaluation index of machine-harvested cotton defoliation effect provided by embodiment 1 of the invention.

FIG. 2 is a schematic diagram showing the monitoring and evaluation method for comprehensive evaluation index of machine-harvested cotton defoliation effect provided by embodiment 1 of the invention.

FIG. 3 is a schematic diagram showing the network structure of the extreme learning machine model provided by embodiment 1 of the invention.

FIG. 4 is a schematic diagram showing the relationship between the estimated and measured values of the boll-opening rate of the machine-harvested cotton defoliation effect comprehensive evaluation model provided by embodiment 1 of the invention.

FIG. 5 is a schematic diagram showing the structure of monitoring and evaluation system for comprehensive evaluation index of machine-harvested cotton defoliation effect provided by embodiment 2 of the invention.

### 5. SPECIFIC EMBODIMENT OF THE INVENTION

To make the technical solutions provided by the invention more comprehensible, a further description of the invention is given below in combination with the attached drawings and embodiments, and the embodiments are exemplary and not the limitations of the scope of the disclosure. Apparently, the described drawings are merely some embodiments of the application rather than all the embodiments of the application. It should be understood that the application is not limited to the drawings described herein. Based on the drawings in the invention, all other drawings obtained by those of ordinary skill in the art without making creative labor fall within the scope of protection of the invention.

The terminology used herein is for the purpose of describing example embodiments only and is not intended to be limiting. As used herein, the singular forms ‘a’, ‘an’, and ‘the’ may be intended to include the plural forms as well, unless the context clearly indicates otherwise. It will be further understood that the terms ‘comprise’, ‘comprises’, and/or ‘comprising’, ‘include’, ‘includes’, and/or ‘including’, when used in this specification, specify the presence of stated features, steps and elements, but do not preclude the presence or addition of one or more other features, steps and elements thereof.

Although this disclosure makes a variety of references to certain modules in the system according to some embodiments of the present disclosure, however, any number of different modules are used and run on the client and/or server. The modules are illustrative only, and different aspects of systems and methods may use different modules.

In the disclosure, flowcharts are used to illustrate the operations performed by the system according to some embodiments of the present disclosure. It should be understood that the preceding or following operations are not necessarily performed accurately in sequence. On the contrary, various operations may be also performed in reverse or simultaneously as required. Other operations may be added to these processes or removed from these processes.

Cotton is one of China's main cash crops and the most critical fiber crop for the textile industry. Cotton production plays an essential role in international trade and national security and is also an important economic source for cotton farmers. China is one of the world's leading cotton-growing countries, with a national cotton planting area of 319,900 hectares in 2020. To reduce production costs, relieve farmers' labor burden and improve the efficiency of cotton harvesting, the area planted with machine-harvested cotton has been gradually expanded in recent years. Spraying defoliant is a critical technology for machined-harvested cotton, which can promote cotton leaf shedding and boll opening, effectively reducing impurities in the machine-harvested process, improving harvesting efficiency and quality. In addition, the ripening ingredients containing in the defoliants can promote the opening of the cotton boll. In Xinjiang cotton area, due to the low temperature in the late stage of fertility, it is difficult to achieve a high boll-opening rate for a short period of time, thus promoting boll-opening by spraying chemical additives. Different defoliant spraying time and defoliant concentration and other factors have different effects on cotton defoliation effect. With the increasing acreage of machine-harvested cotton, there has been a gradual increase in research on the effect of cotton defoliation.

In agricultural production, the defoliation rate and boll-opening rate are used as the basis for cotton harvesting, and it is generally considered that machine-harvested cotton can be mechanically harvested when the defoliation rate reaches more than 90% and the boll-opening rate reaches more than 95%. Therefore, it is critical to monitor the defoliation rate, boll-opening rate and yield of machine-harvested cotton quickly and accurately, both for machine-harvested cotton defoliant-related research and for determining the harvesting time of machine-harvested cotton in field production. As the machine-harvested cotton leaves shed and the boll opens, the color and texture features of its canopy RGB images change significantly, with green information gradually decreasing and white information gradually increasing. Domestic and foreign scholars have used various methods to analyze the color and texture feature changes of crop canopies RGB images to carry out monitoring of crop leaf growth-related index. Remote sensing technology can achieve timely, dynamic and macroscopic monitoring, to become an important tool for monitoring crop growth information.

In recent years, with the continuous development of remote sensing technology, a large number of domestic and foreign research based on remote sensing technology for crop growth monitoring. Currently, the common remote sensing means include handheld spectrometer, UAV and satellite. Ground-based spectral monitoring has the advantages of non-destructive and accurate, but its spatial scale is smaller and more suitable for mechanistic research due to the limitation of the shooting range and the weight of the instrument. Satellite remote sensing technology has become an important means to collect various agricultural production data and has a certain potential in crop growth monitoring. However, because image resolution of satellite remote sensing technology is mostly in the range of 10-60 m, it is more suitable for monitoring on a large regional scale. Furthermore, the use of satellite sensors has the defects of high cost, low spatial resolution, long sampling period, and cloud interference in image quality. UAV low-altitude remote sensing platforms are becoming popular in the development of precision agriculture, with fast and repeatable capture capability. Currently, UAVs can carry more sensors, such as hyperspectral, thermal imaging, RGB and LiDAR. Compared with satellite remote sensing, UAV remote sensing platform has high flexibility, low cost, and low atmospheric impact, relatively high spatial and temporal resolution, which is more adaptable to small plot monitoring. Among the information available to UAVs, digital images are the most easily accessible and common image information in our daily life, with low cost of information acquisition, and are widely used for crop growth monitoring. Among the sensors carried by UAVs, RGB cameras have the advantages of small size, high resolution and simple operation of information acquisition. RGB images can record the brightness (DN value) of red, green and blue bands, to perform color space conversion, calculate vegetation indices and extract texture features and based on RGB images. Compared with spectral images or multi-source data fusion, RGB images have small data volume and simple process. High-resolution RGB images can be acquired by UAV to fully exploit the image information, which is more conducive to reducing monitoring costs and complexity.

The purpose of the invention is to provide a monitoring and evaluation method for comprehensive evaluation index of machine-harvested cotton defoliation effect and system thereof, to improve the accuracy and efficiency of monitoring and evaluating the comprehensive evaluation index of machine-harvested cotton defoliation effect, and to provide reference for studying the machine-harvested cotton defoliation effect and determining the optimal harvesting time of machine-harvested cotton.

The various features and advantages of this invention and the manner of attaining them will become more apparent and the invention itself will be better understood by reference to the following description of embodiments of the invention taken in conjunction with the accompanying drawings.

### Embodiment 1

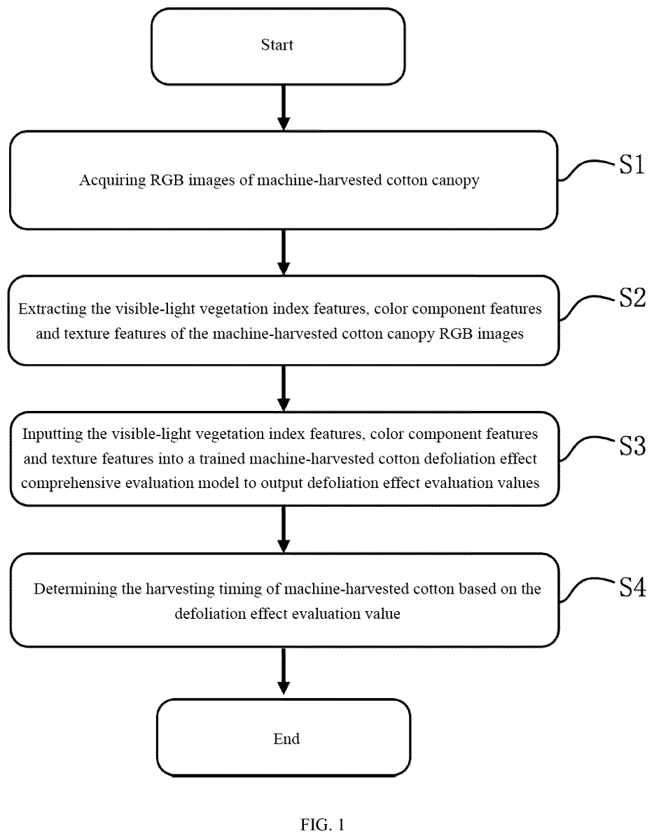

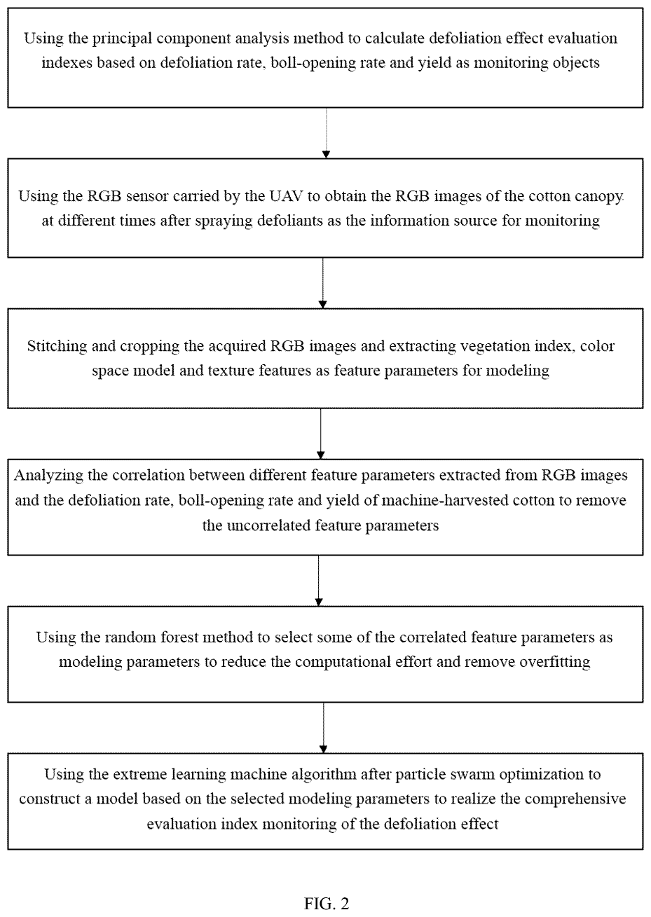

As shown in FIG. 1 and FIG. 2, the embodiment provides a monitoring and evaluation method for comprehensive evaluation index of machine-harvested cotton defoliation effect, comprising the following steps:

Step 1. Acquiring RGB images of machine-harvested cotton canopy.

For acquiring RGB images of the machine-harvested cotton canopy in this embodiment, a DJI Phantom 4 Advanced aerial drone is used to acquire images of machine-harvested cotton canopy between 12:00 and 16:00, for the day before and every 3 days after defoliant spraying, up to the 15th day. The relevant parameters of the drone on the day before harvest are shown in Table 1:

TABLE 1
Phantom 4 Advanced Aerial Photography Drone Parameters

| Parameters | Value | |
|---|---|---|
| Drone Weight | 1368 | g |
| Maximum Flight Time | 30 | min |
| Sensor | 1-inch CMOS, 20 million | |
| | effective pixels | |
| Ground Resolution | 0.3 | cm |

| | |
|---|---|
| Focal Length | 24 |
| Spectral Band | R, G, B |

This embodiment establishes the overlap rate between adjacent routes as 80% and the overlap rate between adjacent pictures on the heading as 80% according to the aerial image stitching requirements. According to the image resolution requirements and flight software limitations, the flight altitude is set to 10 meters when acquiring images. After determining the flight altitude and overlap rate, the shooting areas are set on DIJ GO GSP software, with the lens vertical downward during image acquisition. The exposure time and ISO are adjusted to the fixed values according to the weather at the time of shooting, and the automatic shooting is carried out after route planning to acquire RGB images of the machine-harvested cotton canopy.

In this embodiment, the size of the acquired machine-harvested cotton canopy RGB image is 5472×3648 pixels, and the format is JPG. After the acquisition of the machine-harvested cotton canopy RGB image, the machine-harvested cotton canopy RGB image is also stitched and cropped by Pix4Dmapper software. The software automatically recognizes the GPS information of the image, to acquire RGB ortho-image of the machine-harvested cotton canopy after completing the GPS information processing, stored in TIFF format and retaining the grayscale information of the three colors of red (R), green (G) and blue (B) of the feature, with values ranging from 0 to 255.

In this embodiment, before cotton is sprayed with defoliant, 20 cotton plants with uniform and representative growth are selected in each treatment plot, and the number of leaves, boll opening and green boll are surveyed before and after 3, 6, 9, 12 and 15 days, respectively. The number of drone shots is the same as the number of surveys, and the defoliation rate and boll-opening rate are calculated based on the survey results.

The formula for calculating defoliation rate and boll-opening rate is as follows:

$$Defoliation rate = [ ( number of leaves of cotton plant before defoliant - number of leaves remaining at the time of survey ) / number of leaves of cotton plant before defoliant ] \times 100 \%$$

$$Boll ‐ opening rate = ( number of boll ‐ opening / total number of boll ) \times 100 \%$$

It should be noted that the invention does not limit the model, parameters, acquisition time, acquisition period, and RGB image size and format of the UAV, which can be set according to the actual situation.

FIG. 2 is a schematic diagram showing the monitoring and evaluation method for comprehensive evaluation index of machine-harvested cotton defoliation effect provided by embodiment 1 of the invention, wherein comprises the process of determining defoliation effect evaluation index using principal component analysis. Therefore, this embodiment comprises the following steps prior to the step of acquiring RGB images of machine-harvested cotton canopy:

Step A1. Collecting historical base data of machine-harvested cotton in the area to be monitored, which means that the field surveys are conducted in the area to be monitored to collect historical basic data of the area, including historical data of defoliation rate, boll-opening rate and yield.

Step A2. Using principal component analysis (PCA) to determine a comprehensive evaluation index of the machine-harvested cotton defoliation effect according to the historical base data. The comprehensive evaluation index of the machine-harvested cotton defoliation effect referring to the index for evaluating the harvesting timing of machine-harvested cotton. The comprehensive evaluation index of the machine-harvested defoliation effect comprises defoliation rate, boll-opening rate and yield.

The invention performs principal component analysis for defoliation rate, boll-opening rate and yield under different spraying time and spraying concentration conditions. Principal component analysis is a data dimensionality reduction algorithm, to map n-dimensional features to k-dimensions, which means to convert an n×m matrix into an n×k matrix, retaining only the main characteristics present in the matrix to greatly save space and data volume.

Step A2 specifically comprises:
Step A2.1. Performing the normalization of the historical base data, which means subtracting the mean of the corresponding variable and dividing by its square deviation, to obtain a data matrix corresponding to the historical base data, and the normalization shown in formula (1):

$${X}_{ij}^{′} = \frac{{X}_{ij} - {\overline{X}}_{j}}{{S}_{j}} , ( j = 1 , 2 , 3 , … , m ) \qquad ( 1 )$$

where X̅ⱼ denotes the mean of the corresponding variable, Sⱼ denotes the square deviation of the corresponding variable, Xᵢⱼ′ denotes the standardized variable, and Xᵢⱼ denotes the variable before standardization, thus generating the standardized data matrix X.
Step A2.2. Calculating the correlation matrix RX or the covariance matrix Cov(X) corresponding to the data matrix X based on the data matrix X.
Step A2.3. Determining the eigenvalues of the correlation matrix or covariance matrix and calculating the eigenvectors corresponding to each eigenvalue.

Taking the correlation matrix RX as an example, the characteristic formula of the correlation matrix RX is used to find the m non-negative eigenvalues, and these eigenvalues are arranged in order from largest to smallest, and then formula (2) can be obtained:

$$\{ \begin{matrix}( {R}_{x} - {λ}_{iI} ) & {α}_{i} = 0 \\ {α}_{i}^{′} & {α}_{i} = 1\end{matrix} ( i = 1 , 2 , … , m ) \qquad ( 2 )$$

where λ_{I} denotes the eigenvalue of the correlation matrix, λ_{iI} denotes the i^{th} eigenvalue of the correlation matrix, αᵢ denotes the eigenvector of the corresponding index, and αᵢ′ is the reciprocal of αᵢ. The eigenvector λ_{iI} of the index corresponding to each i^{th} eigenvalue is solved to determine each principal component according to formula (3):

$${X}^{′} = {α}_{i 1} {X}_{1} + {α}_{i 2} {X}_{2} + … + {α}_{i m} {X}_{m} \qquad ( 3 )$$

Where, X′ denotes the principal component value which means the principal component score, Xₘ denotes the m^{th} index; αᵢₘ denotes the corresponding i^{th} eigenvector of the m^{th} index.
Step A2.4. Determining the principal component eigenvectors based on the eigenvectors and calculating the contribution and cumulative contribution of the principal component eigenvectors.
where the contribution rate of the i^{th} principal component is calculated by formula (4):

$$\frac{{λ}_{i}}{{\sum }_{i = 1}^{m} {λ}_{i}} = \frac{{λ}_{i}}{m} \qquad ( 4 )$$

where λᵢ denotes the i^{th} principal component feature and m denotes the number of features.

The cumulative contribution rate of the first k principal components is calculated as formula (5):

$${\sum }_{i = 1}^{k} \frac{{λ}_{i}}{m} \qquad ( 5 )$$

Step A2.5. Determining the comprehensive evaluation index of machine-harvested cotton defoliation effect based on the contribution rate and cumulative contribution rate of the principal component eigenvector, comprising defoliation rate, boll-opening rate and yield.

Calculating the contribution and cumulative contribution of the principal component eigenvectors based on principal component analysis is shown in the table 2.

TABLE 2
Contribution and Cumulative Contribution of the Principal
Component Eigenvectors

| | Component | | |
|---|---|---|---|
| Item | PCA1 | PCA2 | PCA3 |
| Defoliation Rate | 0.3207 | 0.9217 | 0.2183 |
| Boll-opening Rate | −0.6459 | 0.3814 | −0.6613 |
| Yield | 0.6928 | 0.0711 | 0.7177 |
| Eigenvalue | 1.9999 | 0.9312 | 0.0690 |
| Contribution Rate (%) | 96.51 | 3.48 | 0.00 |
| Cumulative Contribution Rate (%) | 96.51 | 99.99 | 100.00 |

As shown in the Table 2, the contribution of one component PCA1 can reach 96.51%, proving that more than 95% of the defoliation effect information of the original data can be achieved by using PCA1, which can be used to represent defoliation rate, boll-opening rate and yield as comprehensive evaluation indexes. In this embodiment, PCA1 is defined as a standard threshold value for comprehensive evaluation of the machine-harvested cotton, and its value is determined by the comprehensive evaluation indexes of defoliation rate, boll-opening rate and yield.
Step A3. Calculating the standard threshold value of machine-harvested cotton defoliation effect comprehensive evaluation according to the machine-harvested cotton defoliation effect comprehensive evaluation index; the standard threshold value of machine-harvested cotton defoliation effect comprehensive evaluation referring to the standard threshold value for evaluating the harvesting timing of machine-harvested cotton; determine the suitability of the machine-harvested cotton for harvesting corresponding to the machine-harvested cotton canopy RGB image.

Table 3 shows the score coefficient matrix of each component, and the composite score of PCA1 can be calculated based on the component score coefficient matrix.

TABLE 3
Principal Component Score Coefficient Matrix

| | PCA1 | PCA2 | PCA3 |
|---|---|---|---|
| Defoliation Rate | 0.7882 | 0.0008 | 0.0000 |
| Boll-Opening | 0.1023 | 0.0121 | 0.9879 |
| Rate | | | |
| Yield | 0.1095 | 0.9871 | 0.0121 |

In this embodiment, according to the comprehensive evaluation index of machine-harvested cotton defoliation effect, the comprehensive evaluation standard threshold of machine-harvested cotton defoliation effect is calculated by the following formula (6):

$$PCA 1 = 0.9992 \times T + 0.0008 \times C \qquad ( 6 )$$

wherein, PCA1 indicates the standard threshold value of machine-harvested cotton defoliation effect comprehensive evaluation, T indicates defoliation rate, and C indicates yield.

It should be noted that the invention adopts defoliation rate, boll-opening rate and yield as comprehensive evaluation indexes and thus predetermines the comprehensive evaluation standard threshold value for the machine-harvested cotton defoliation effect. Since the coefficient of boll-opening rate is approximately equal to 0 after extensive tests, only the defoliation rate and yield are used to simplify the calculation process when PCA1 is used for the comprehensive evaluation of the machine-harvested cotton defoliation effect. However, the machine-harvested cotton timely harvesting standard stipulates that the defoliation rate reaches 90% or more, while the boll-opening rate reaches 95% or more can be harvested. Therefore, in practical application, the boll-opening rate should be regarded as an important index to evaluate the machine-harvested cotton defoliation effect.
Step 2. Extracting the visible-light vegetation index features, color component features and texture features of the machine-harvested cotton canopy RGB images, specifically comprising:
Step 2.1. Dividing the RGB ortho-images of the of machine-harvested cotton canopy according to the location of each test plot in the area to be monitored, to obtain a plurality of regions of interest (ROI).
Step 2.2. Obtaining the digital number (DN) of each color channel in each region of interest and calculating the average digital quantization value of each color channel; and the color channels comprising R channel, G channel and B channel.

The information of RGB ortho-image comprises the DN of three color channels: red, green and blue. This embodiment acquires the DN of three color channels for each region of interest using MATLAB 2019a software and calculates the average DN of each color channel.
Step 2.3. Performing normalization of the digital quantization value and the average digital quantization value of each color channel and calculating each color component value; the color component value referring to the normalized value of each color component in the RGB ortho-image, and the RGB ortho-image each color component comprising the r component, the g component and the b component.
Step 2.4. Calculating the visible-light vegetation index features based on the respective color component values;

In this embodiment, the original DN of the three color channels R channel, G channel and B channel is divided by the sum of the DN of the three color channels, and the normalized values r, g and b of the three color components r, g and b are calculated as shown in formulas (7), (8) and (9), respectively:

$$r = \frac{R}{( R + G + B )} \qquad ( 7 )$$

$$g = \frac{G}{( R + G + B )} \qquad ( 8 )$$

$$b = \frac{B}{( R + G + B )} \qquad ( 9 )$$

The visible-light vegetation index features selected in this embodiment include NGRDI, MGRVI, RGBVI, NDI, VARI, WI, CIVE, GLA, ExG, ExR, ExGR, GLI, and NGBDI, and the corresponding color component features in color space include R, G, B, r, g, b, Y, Cb, Cr, and U. The calculation formulas for each visible-light vegetation index feature and color component feature are shown in Table 4:

TABLE 4
Expressions for the Color Space and Visible-light Vegetation Index

| | Color Component | |
|---|---|---|
| | Band and | |
| Data Type | Vegetation Index | Formula |
| Color | R | DN of Red Light Band |
| Space | G | DN of Green Light Band |
| | B | DN of Blue Band |
| | r | $r = \frac{R}{( R + G + B )}$ |
| | g | $g = \frac{G}{( R + G + B )}$ |
| | b | $b = \frac{B}{( R + G + B )}$ |
| | Y | Y = 0.299R + 0.587G + 0.114B |
| | Cb | Cb = 0.568 × (B − Y) + 128 |
| | Cr | Cr = 0.713 × (R − Y) + 128 |
| | U | U = 0.493 × (B − Y × 2) |
| Vegetation Index | NGRDI | $NGRDI = \frac{( g - r )}{( g + r )}$ |
| | MGRVI | $MGRVI = \frac{( {g}^{2} - {r}^{2} )}{( {g}^{2} + {r}^{2} )}$ |
| | RGBVI | $MGBVI = \frac{( {g}^{2} - br )}{( {g}^{2} + br )}$ |
| | NDI | $NDI = \frac{( r - g )}{( r + g + 0.01 )}$ |
| | VARI | $VARI = \frac{( g - r )}{( g + r - b )}$ |
| | WI | $WI = \frac{( g - b )}{( r - g )}$ |
| | CIVE | CIVE = 0.441r − 0.881g + 0.385b + 18.78745 |
| | GLA | $GLA = \frac{( 2 G - B - R )}{( 2 G + B + R )}$ |
| | ExG | ExG = 2g − b − r |
| | ExR | ExR = 1.4r − g |
| | ExGR | ExGR = 3g − 2.4r − b |
| | GLI | $GLI = \frac{( 2 g - b - r )}{( 2 g + b + r )}$ |
| | NGBDI | $NGBDI = \frac{( g - b )}{( g + b )}$ |

Step 2.5. Performing the color space model transformation on the RGB color space model corresponding to the color features in each region of interest, respectively, to obtain a transformed color space model; the transformed color space model comprising an HSV color space model, a La*b* color space model, a YCrCb color space model, and a YIQ color space model.
Step. 2.6. Extracting color component features in each color space model according to the transformed color space model and calculating the digital number of each the color component feature.

In this embodiment, the color features of each divided region of interest are converted from RGB color space model to HSV color space model, La*b* color space model, YCrCb color space model and YIQ color space model, where the HSV color space model, La*b* color space model and YIQ color space model are calculated based on the rgb2hsv function, rgb2lab function and rgb2ntsc function in MATLAB, and the conversion formulas of other parameters are shown in Table 4.
Step. 2.7. Calculating the texture features of angular second moment, entropy, contrast and correlation based on the gray-level co-occurrence matrix.

Ultra-high-resolution images (ground resolution 0.3 cm) are acquired from a UAV flying at an altitude of 10 meters. Four texture features calculated from four different angles (0°, 45°, 90° and) 135° based on the gray-level co-occurrence matrix (GLCM) are selected to calculate the mean and square deviation (sd) of each angle of the four texture features. The three bands of the RGB image are calculated as gray-level values before calculating the texture features, as shown in Table 5.

The texture features used in this implementation include the angular second moment texture feature, entropy texture feature, contrast texture feature and correlation texture feature, and the meaning and calculation formula of each texture feature are as follows:
(1) Angular Second Moment (Asm) texture feature: indicating the change of image energy value, reflecting the uniformity of image gray value distribution and texture thickness; when all pixels in the image have the same gray value, the energy value is 1, and the calculation formula is formula (10):

$$A s m = {\sum }_{i} {\sum }_{j} {P ( i , j )}^{2} \qquad ( 10 )$$

wherein, P(i, j) denotes the gray value corresponding to the pixel point with horizontal coordinate i and vertical coordinate j.
(2) Entropy (Ent) texture features: reflecting the complexity of the gray value distribution in the image, and the more considerable Ent value reflects the more complex pixel distribution in the image and the more dispersed distribution of the same elements, calculated as formula (11):

$$E n t = {\sum }_{i} {\sum }_{j} P ( i , j ) log P ( i , j ) \qquad ( 11 )$$

(3) Contrast (Con) texture feature: reflecting the clarity and texture depth of the image. The deeper texture reflects the larger Con and the more precise image, and the more enormous variation of gray value between pixels, which is calculated by formula (12):

$$C o n = {\sum }_{i} {\sum }_{j} {P ( i , j )}^{2} P ( i , j ) \qquad ( 12 )$$

(4) Correlation (Cor) texture feature: reflecting the predictable linear relationship between the gray values of two adjacent pixels in the window, and the larger the Cor reflects the greater predictability between pixels and the more uniform gray values, calculated as formula (13):

$$C o r = \frac{{\sum }_{i} {\sum }_{j} P ( i , j ) P ( i , j ) - {μ}_{x} {μ}_{y}}{{σ}_{x} {σ}_{y}} \qquad ( 13 )$$

In this embodiment, Pₓ(i)=Σⱼ₌₁ P(i, j), P_{y}(j)=Σᵢ₌₁ P(i, j), wherein μₓ and σₓ denote the mean and square deviation of gray values Pₓ(i) (i=1, 2, . . . , G); μ_{y} and σ_{y} denote the mean and square deviation of gray values P_{y}(j) (j=1, 2, . . . , G), where G denotes the image gray level.

TABLE 5
Extracted Texture Features and Extraction Angles

| Texture Features Angle | Waveband | Angle |
|---|---|---|
| Angular Second Moment (ASM) | Gary | 0°, 45°, 90°, 135° |
| Entropy (Ent) | | |
| Contrast (Con) | | |
| Correlation (Corr) | | |

It should be noted that the invention does not limit the specific categories of visible-light vegetation index features, color component features and texture features, and each of the above-mentioned visible-light vegetation index features, color component features and texture features is only an exemplary description, and other visible-light vegetation index features, color component features and texture features can be included, which should be set according to the actual machine-harvested cotton canopy RGB images.

In this embodiment, after extracting the visible-light vegetation index features, color component features and texture features from the RGB images of the machine-harvested cotton canopy, the correlation between different feature parameters such as visible-light vegetation index features, color component features and texture features extracted from the RGB images of the machine-harvested cotton canopy and the defoliation rate, boll-opening rate and yield of machine-harvested cotton respectively can be analyzed. The irrelevant or poorly correlated feature parameters can be removed, simplifying the calculation process and ensuring the accuracy of the comprehensive evaluation model of the machine-harvested cotton defoliation effect as much as possible.

Furthermore, the embodiment further comprises the following step of selecting features such as visible-light vegetation index features, color component features, and texture features, after extracting the visible-light vegetation index features, color component features and texture features of the machine-harvested cotton canopy RGB images:
selecting the extracted visible-light vegetation index features, color component features and texture features using random forest (RF) method respectively to obtain the selected image features; the selected image features comprising at least one visible-light vegetation index feature, at least one color component feature, and at least one texture feature.

Selecting some of the features as modeling parameters from the feature parameters having correlation by the random forest method reduces the computational effort and eliminates the overfitting problem. Furthermore, the selected image features include at least one visible-light vegetation index feature, at least one color component feature and at least one texture feature to ensure that the machine-harvested cotton defoliation effect can be evaluated by combining the visible-light vegetation index, color component and texture features of the RGB images of the machine-harvested cotton canopy from the three feature dimensions of visible-light vegetation index, color component and texture features, to improve the accuracy of the evaluation results.

In this embodiment, 39 feature information (mean and square deviation of 4 texture features, 18 color components and 13 visible-light vegetation indexes) extracted from the high-resolution RGB images acquired by UAV are targeted. Based on the random forest method, the 10 parameters with the highest contribution rate are selected as modeling objects, respectively, and a comprehensive evaluation model of the machine-harvested cotton defoliation effect is constructed and trained to facilitate the direct real-time acquisition of machine-harvested cotton canopy RGB images for practical applications. Furthermore, the extracted visible-light vegetation index, color components and texture features are directly input into the pre-trained comprehensive evaluation model of the machine-harvested cotton defoliation effect, to output the corresponding defoliation effect evaluation value quickly.

Random forest is an integrated machine learning algorithm that uses both bootstrap and node classification techniques for resampling to construct multiple uncorrelated decision trees and generates the final classification results by voting. The random forest method can analyze the relationship between features with complex interactions and has good robustness to noisy and missing data with fast learning speed, where variable importance can be used as the basis for feature selection of high-dimensional data.

The two objectives of the invention for feature selection using the random forest method are to select highly dependent feature variables and to select feature variables with low dimensionality that better express the prediction results. In the random forest method, the Gini index and the error rate of OOB data are usually used to measure the importance of the selected features. The feature selection results are shown in Table 6:

TABLE 6
Feature Selection Results

| Monitoring | Selection | |
|---|---|---|
| Objects | Method | Selection Results |
| PCA1 | RF | Con-sd, Q, Con-mean, Ent-sd, Cor-sd, S, |
| | | NGRDI, NDI, MGRVI, Cr |

As shown in Table 6, three vegetation index features, three color component features and four texture features are selected based on the random forest method, among which, the highest contribution is the texture feature Con-sd and the lowest contribution is the color component Cr.
Step 3. Inputting the visible-light vegetation index features, color component features and texture features into a trained machine-harvested cotton defoliation effect comprehensive evaluation model to output defoliation effect evaluation values.

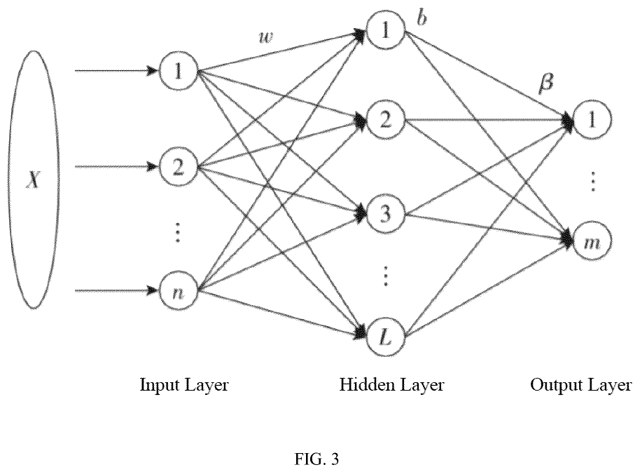

The trained comprehensive evaluation model of machine-harvested cotton defoliation effect is an extreme learning machine (ELM) model based on particle swarm optimization (PSO) algorithm through training with visible-light vegetation index features, color component features and texture features of the machine-harvested cotton canopy RGB images as input and the defoliation effect evaluation value as output.

Wherein, the extreme learning machine is a single hidden layer feedforward neural network, with a faster learning speed compared to the traditional feedforward neural network. The extreme learning machine comprises an input layer, a hidden layer and an output layer, as shown in FIG. 3. In contrast to the traditional neural network algorithm, the goal of the extreme learning machine is to achieve the minimum training error and the minimum output weight parametrization. The weights of the hidden layer can be generated randomly without iterative optimization, which is suitable for real-time training. Moreover, extreme learning machines can handle complex data and enable robustness for multiple highly correlated variables. The particle swarm optimization algorithm is a computational method that simulates the foraging behavior of birds and finds the optimal solution through collaboration and information sharing among individuals in a population.

The comprehensive evaluation model of the machine-harvested cotton defoliation effect used in the invention is the PSO-ELM model, and these feature parameters are input to the trained PSO-ELM model after selecting three vegetation indexes, three color components and four texture features. Wherein, the extreme learning machine model comprises an input layer, a hidden layer and an output layer, and the particle swarm optimization algorithm described is used to optimize the weight values of the input layer and the bias values of the hidden layer, which can reduce the number of hidden nodes required for each layer of the extreme learning machine and improve the generalization ability of the network after training. Parameters such as inertia weights, learning factor, the maximum number of iterations and population size are considered during the optimization of the particle swarm optimization algorithm.

The process of the particle swarm optimization algorithm comprises:
(1) Initialization. Assuming that there are n particles in the swarm, the particle swarm is initialized, and each particle is assigned a random initial position and velocity;
(2) Adaptation value calculation. Calculating the adaptation value of each particle according to the adaptation function;
(3) Individual best adaptation value calculation. Comparing the adaptation value of its current position with the adaptation value corresponding to its historical best position for each particle, and updating the historical best position with the current position if the adaptation value of the current position is higher;
(4) Obtaining the population's best adaptation value. Comparing the adaptation value of its current position with the adaptation value corresponding to its global best position for each particle, and updating the global best position using the current position if the adaptation value of the current position is higher;
(5) Calculating and updating the velocity and position of each particle. The calculation of the velocity and position of the particle is the same as the prior art and is not repeated herewith.
(6) Determining the completion of the algorithm. If the end condition is not satisfied, the algorithm returns to step (2), and if the end condition is satisfied, the algorithm is completed, and the global best position is the global optimal solution.

In the stage of establishing and training the model, the invention acquires sample data based on UAV and ground to obtain a total of 335 sets of samples (each set of samples includes 10 feature parameters selected by Step 2 and the corresponding PCA1), which are randomly divided into 201 samples as the training set and 134 samples as the validation set. The mean square error between the predicted and observed values of the test samples is used as the fitness of the particle swarm optimization algorithm to calculate the individual extremes and global extremes, and the positions and velocities of the particles are updated by iteration of the fitness of the particle swarm optimization algorithm.

The invention uses the strategy of reducing the adaptive inertia weights, setting the maximum value of the inertia weights in the particle swarm optimization algorithm in the range from 1 to 2.5 to increase the probability of finding the global optimal peak, and the minimum value is chosen in the range from −1 to −2.5 to allow the particles to converge to the optimal value slowly. The final maximum and minimum values of inertia weights are set to 1 and −1, respectively. The two learning rates are acceleration constants, and the best learning rate is found by a test with a step size of 0.1, which is finally set to 1.4945 and 1.3128. The maximum number of iterations is set to 50, and the population size is tested from 15 to 200 with a step size of 15. Then the individual extremes and global extremes of the particles are updated until the minimum error is obtained, or the maximum number of iterations is reached. Finally, the input layer weights, and hidden layer bias values of the generated optimal results are used as input parameters for the extreme learning machine model.

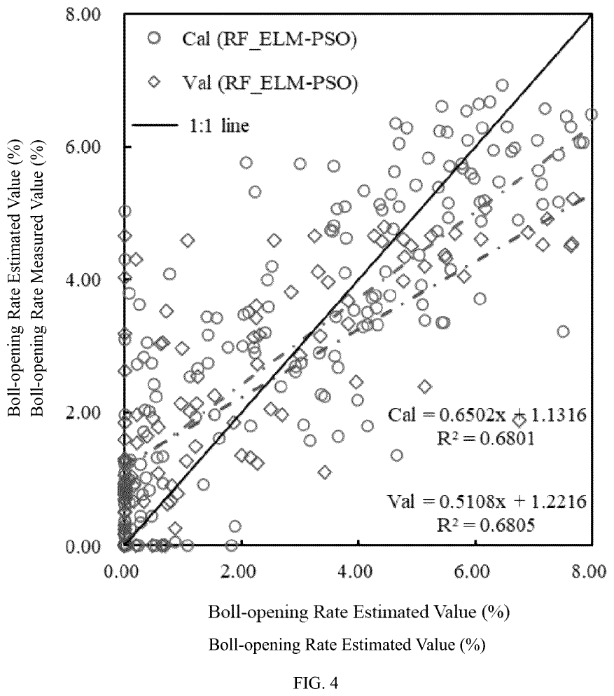

The results are shown in Table 7, with R²=0.6801, RMSE=1.5754, and rRMSE=51.08% for the PSO-ELM model training set, and R²=0.6805, RMSE=1.6257, and rRMSE=56.90% for the validation set. FIG. 4 illustrates the linear relationship between the true and predicted values of each model training set (Cal) and validation set (Val), where PF_PSO-ELM represents the extreme learning machine model based on the random forest method and particle swarm optimization algorithm used in the invention, which is the comprehensive evaluation model of the machine-harvested cotton defoliation effect. As shown in FIG. 4, the linear trend between the predicted and measured values of the model is close to the 1:1 line, which proves that the model can be used for practical applications.

TABLE 7
Comprehensive Evaluation Model of Machine-Harvested Cotton Defoliation Effect

| Modeling | Selection | Training Set | | | Validation Set | | |
|---|---|---|---|---|---|---|---|
| Methods | Methods | R² | RMSE | rRMSE (%) | R² | RMSE | rRMSE (%) |
| PSO-ELM | RF | 0.6801 | 1.5754 | 51.08 | 0.6805 | 1.6257 | 56.90 |

The invention adopts an extreme learning machine model based on particle swarm optimization algorithm as a comprehensive evaluation model of machine-harvested cotton defoliation effect, and takes the visible-light vegetation index features, color component features and texture features of machine-harvested cotton canopy RGB images as model inputs, to truly and effectively reflect the machine harvested cotton defoliation effects. Therefore, the invention can accurately and reliably determine the best harvesting time of machine-harvested cotton, improve the accuracy of monitoring and evaluation of the comprehensive evaluation index of machine-harvested cotton defoliation effect, solve the problem of strong subjectivity and low accuracy of the traditional method, to provide estimation technical support for the research related to the machine-harvested cotton defoliation effect, and to provide reference for determining the best harvesting time of machine-harvested cotton in agricultural production.
Step 4. Determining the harvesting timing of machine-harvested cotton based on the defoliation effect evaluation value.

The invention obtains the defoliation effect evaluation value in Step 3 and uses the defoliation effect evaluation value to evaluate the machine-harvested cotton defoliation effect of the machine-harvested cotton corresponding to the RGB images of the machine-harvested cotton canopy, to determine the suitability of the machine-harvested cotton for harvesting. Specifically comprises:
comparing the size of the defoliation effect evaluation value with the standard threshold value of machine-harvested cotton defoliation effect comprehensive evaluation, and determining the suitability of the machine-harvested cotton for harvesting corresponding to the machine-harvested cotton canopy RGB image based on the comparison result, with the following two determinations:
(1). Determining that the machine-harvested cotton corresponding to the machine-harvested cotton canopy RGB image is suitable for harvesting, when the defoliation effect evaluation value is greater than the threshold value of the machine-harvested cotton defoliation effect comprehensive evaluation;
(2). Determining that the machine-harvested cotton corresponding to the machine-harvested cotton canopy RGB image is not suitable for harvesting, when the defoliation effect evaluation value is less than or equal to the threshold value of the machine-harvested cotton defoliation effect comprehensive evaluation.

In this embodiment, according to the machine-harvested cotton timely harvesting standards: the cotton with a defoliation rate of 90% or more and a boll-opening rate of 95% or more can be harvested. Taking the Xinjiang area of machine-harvested cotton as an example, the yield data are obtained from the cotton production in the statistical yearbook of Xinjiang and other corresponding areas. This embodiment refers to the statistical yearbook of Xinjiang region cotton average seed yield of 360 kg/mu, which can be calculated into the formula (6) to obtain a comprehensive evaluation of the machine-harvested cotton defoliation effect standard threshold value PCA1, and the PCA1 in this embodiment is 1.3225. It is understandable that the value of 1.3225 for PCA1 is only an exemplary description of the comprehensive evaluation threshold of the machine-harvested cotton defoliation effect in this invention, and the value of PCA1 is not fixed and unique and should be determined according to the actual situation of defoliation rate, boll-opening rate and yield in the area to be tested.

After inputting the visible-light vegetation index features, color component features and texture features to the trained machine-harvested cotton defoliation effect comprehensive evaluation model in Step 3, a defoliation effect evaluation value PCAₚ is output. The defoliation effect evaluation value PCAₚ is compared with the standard threshold value PCA1 for comprehensive evaluation of the machine-harvested cotton defoliation effect, and the result of the comparison can directly determine whether the machine-harvested cotton defoliation effect corresponding to the currently collected RGB images of the machine-harvested cotton canopy meets the standard and the suitability for harvesting. When the defoliation effect evaluation value PCAₚ>1.3225, it means that the defoliation effect meets the standard and is suitable for harvesting; when PCAₚ≤1.3225, it means that the defoliation effect does not meet the standard and is not suitable for harvesting.

To demonstrate the effectiveness of the method of the invention, the following experiments have been conducted in this embodiment:

During the experiment, a DJI Phantom 4 Advanced aerial drone is used to collect RGB images based on the machine-harvested cotton canopy covering 48 plots, and the visible-light vegetation index, color components and texture features are extracted from the images. An extreme learning machine model based on the random forest method and particle swarm optimization algorithm is used to perform the inversion of the comprehensive evaluation index of the defoliation effect at different times during the harvesting period, and the PCA1 value is used as the criterion to determine the harvesting time. The harvesting can be started when PCAₚ>PCA1, and the discriminative accuracy of PCA1 is calculated by comparing the ground-measured defoliation rate, boll-opening rate and yield processing with the inverse performance of RGB images obtained by UAV. The results showed that when monitoring is performed 10 days before harvest, the range of PCAₚ is from 1.2216-7.1434, and the predicted values obtained by using the comprehensive evaluation model of machine-harvested cotton defoliation effect are overestimated by 1.3225-3.5717 compared with the true values, resulting in the true unharvestable area being determined to be harvestable, while underestimating the part of 5.3576-7.1431. When monitored 5 days before harvest, the range of PCAₚ is from 1.2217 to 9.1019, and the determination regarding the harvestability is basically accurate, and the comprehensive evaluation model of machine-harvested cotton defoliation effect only miscalculated one unharvestable plot as harvestable, while underestimating a higher area. When monitored 1 day before harvest, there is no miscalculation regarding the harvestability, but there remained the large PCAₚ to be underestimated. In conclusion, it is feasible to monitor the comprehensive evaluation index of the defoliation effect and determine the harvesting timing by using the high-resolution machine-harvested cotton canopy RGB images obtained by UAV.

The defoliation rate, boll-opening rate and yield of machine-harvested cotton have a significant role in the field management of machine-harvested cotton and determining the best harvesting time, as a single index to determine the harvesting time is inevitably inaccurate. The invention has essential value for machine-harvested cotton field management and the best harvesting time determination. The invention is based on the defoliation rate, boll-opening rate and yield of the three indexes to establish a comprehensive index and define the determination criteria to determine the comprehensive evaluation of the effectiveness of machine-harvested cotton defoliation criteria threshold. Moreover, the traditional survey method is primarily based on manual, which is challenging to achieve regional judgment without being representative. The invention adopts an extreme learning machine model based on particle swarm optimization algorithm as a comprehensive evaluation model of machine-harvested cotton defoliation effect, and takes the visible-light vegetation index features, color component features and texture features of machine-harvested cotton canopy RGB images as model inputs, to truly and effectively reflect the machine harvested cotton defoliation effects. Therefore, the invention can accurately and reliably determine the best harvesting time of machine-harvested cotton, improve the accuracy of monitoring and evaluation of the comprehensive evaluation index of machine-harvested cotton defoliation effect, solve the problem of strong subjectivity and low accuracy of the traditional method, to provide estimation technical support for the research related to the machine-harvested cotton defoliation effect, and to provide reference for determining the best harvesting time of machine-harvested cotton in agricultural production.

### Embodiment 2

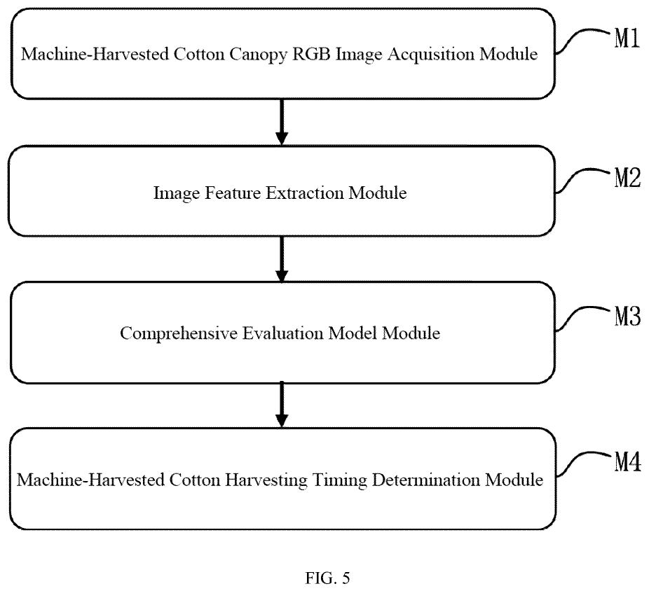

As shown in FIG. 5, the embodiment provides a monitoring and evaluation system for comprehensive evaluation index of machine-harvested cotton defoliation effect, and the function of each module in the system is the same as and corresponds to each step of the method in embodiment 1, comprising:

A machine-harvested cotton canopy RGB image acquisition module M1 for acquiring machine-harvested cotton canopy RGB images.

An image feature extraction module M2 for extracting visible-light vegetation index features, color component features and texture features of the machine-harvested cotton canopy RGB images.

A comprehensive evaluation model module M3 for inputting the visible-light vegetation index features, color component features and texture features into a trained comprehensive evaluation model of machine-harvested cotton defoliation effect to output the defoliation effect evaluation values; the trained comprehensive evaluation model of machine-harvested cotton defoliation effect is an extreme learning machine model based on particle swarm optimization algorithm through training with visible-light vegetation index features, color component features and texture features of the machine-harvested cotton canopy RGB images as input and the defoliation effect evaluation value as output.

A machine-harvested cotton harvesting timing determination module M4 for determining the harvesting timing of machine-harvested cotton based on the defoliation effect evaluation value.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

The invention and the embodiments thereof are described hereinabove, and this description is not restrictive. Although a few embodiments of the invention have been shown and described, it will be understood by those of ordinary skill in the art that a variety of variations, modifications, replacements and variants of these embodiments can be made without materially departing from the novel teachings and advantages of example embodiments. Accordingly, all such modifications are intended to be included within the scope of example embodiments as defined in the claims. Therefore, it is to be understood that the foregoing is illustrative of example embodiments and is not to be construed as limited to the specific embodiments disclosed, and that modifications to the disclosed example embodiments, as well as other example embodiments, are intended to be included within the scope of the appended claims. The inventive concept is defined by the following claims, with equivalents of the claims to be included therein.

1. A monitoring and evaluation method for comprehensive evaluation index of machine-harvested cotton defoliation effect, wherein comprises the following steps:
acquiring RGB images of machine-harvested cotton canopy;
extracting the visible-light vegetation index features, color component features and texture features of the machine-harvested cotton canopy RGB images;
inputting the visible-light vegetation index features, color component features and texture features into a trained machine-harvested cotton defoliation effect comprehensive evaluation model to output defoliation effect evaluation values, the trained comprehensive evaluation model of machine-harvested cotton defoliation effect is an extreme learning machine model based on particle swarm optimization algorithm through training with visible-light vegetation index features, color component features and texture features of the machine-harvested cotton canopy RGB images as input and the defoliation effect evaluation value as output, the extreme learning machine model comprising an input layer, a hidden layer and an output layer, and the particle swarm optimization algorithm provided for optimizing the weight values of the input layer and the bias values of the hidden layer,
determining the harvesting timing of machine-harvested cotton based on the defoliation effect evaluation value.

2. A method as claimed in claim 1, wherein the method further comprises the following step after acquiring the machine-harvested cotton canopy RGB image:
stitching the machine-harvested cotton canopy RGB image by Pix4Dmapper software to obtain a machine-harvested cotton canopy RGB ortho-image.

3. A method as claimed in claim 2, wherein the step of extracting the visible-light vegetation index features, color component features and texture features of the machine-harvested cotton canopy RGB images, specifically comprises:
dividing the RGB ortho-images of the of machine-harvested cotton canopy according to the location of each test plot in the area to be monitored, to obtain a plurality of regions of interest;
obtaining the digital number of each color channel in each region of interest, and calculating the average digital quantization value of each color channel; the color channels comprising R channel, G channel and B channel;
performing normalization of the digital quantization value and the average digital quantization value of each color channel; and calculating each color component value; the color component value referring to the normalized value of each color component in the RGB ortho-image; and the RGB ortho-image each color component comprising the r component; the g component and the b component;
calculating the visible-light vegetation index features based on the respective color component values;
performing the color space model transformation on the RGB color space model corresponding to the color features in each region of interest; respectively; to obtain a transformed color space model; the transformed color space model comprising an HSV color space model; a La*b* color space model; a YCrCb color space model; and a YIQ color space model;
extracting color component features in each color space model according to the transformed color space model; and calculating the digital number of each the color component feature;
calculating the texture features of angular second moments; entropy; contrast and correlation based on the gray-level co-occurrence matrix.

4. A method as claimed in claim 1; wherein the method further comprises the following step after extracting the visible-light vegetation index features; color component features and texture features of the machine-harvested cotton canopy RGB images:
selecting the extracted visible-light vegetation index features; color component features and texture features using random forest method respectively to obtain the selected image features; the selected image features comprising at least one visible-light vegetation index feature; at least one color component feature; and at least one texture feature.

5. A method as claimed in claim 1; wherein the method further comprises the following steps before acquiring RGB images of machine-harvested cotton canopy:
collecting historical base data of machine-harvested cotton in the area to be monitored; using principal component analysis to determine a comprehensive evaluation index of the machine-harvested cotton defoliation effect according to the historical base data; the comprehensive evaluation index of the machine-harvested cotton defoliation effect referring to the index for evaluating the harvesting timing of machine-harvested cotton;
calculating the standard threshold value of machine-harvested cotton defoliation effect comprehensive evaluation according to the machine-harvested cotton defoliation effect comprehensive evaluation index; the standard threshold value of machine-harvested cotton defoliation effect comprehensive evaluation referring to the standard threshold value for evaluating the harvesting timing of machine-harvested cotton; determine the suitability of the machine-harvested cotton for harvesting corresponding to the machine-harvested cotton canopy RGB image.

6. A method as claimed in claim 5; wherein the step of using principal component analysis to determine a comprehensive evaluation index of machine-harvested cotton defoliation effect according to the historical base data; specifically comprises:
performing the normalization of the historical base data to obtain a data matrix corresponding to the historical base data;
calculating the correlation matrix or the covariance matrix corresponding to the data matrix based on the data matrix;
determining the eigenvalues of the correlation matrix or covariance matrix and calculating the eigenvectors corresponding to each eigenvalue;
determining the principal component eigenvectors based on the eigenvectors and calculating the contribution and cumulative contribution of the principal component eigenvectors;
determining the comprehensive evaluation index of machine-harvested cotton defoliation effect based on the contribution rate and cumulative contribution rate of the principal component eigenvector.

7. A method as claimed in claim 5; wherein the step of calculating the standard threshold value of machine-harvested cotton defoliation effect comprehensive evaluation according to the machine-harvested cotton defoliation effect comprehensive evaluation index, specif1cally comprises:
according to the comprehensive evaluation index of machine-harvested cotton defoliation effect, the comprehensive evaluation standard threshold of machine-harvested cotton defoliation effect is calculated by the following formula:
PCA1=0.9992×T+0.0008×C
wherein, PCA1 indicates the standard threshold value of machine-harvested cotton defoliation effect comprehensive evaluation, T indicates defoliation rate, and C indicates yield.

8. A method as claimed in claim 5, wherein the step of determining the harvesting timing of machine-harvested cotton based on the defoliation effect evaluation value, specif1cally comprises:
comparing the size of the defoliation effect evaluation value with the standard threshold value of machine-harvested cotton defoliation effect comprehensive evaluation, and determining the suitability of the machine-harvested cotton for harvesting corresponding to the machine-harvested cotton canopy RGB image based on the comparison result, with the following steps:
determining that the machine-harvested cotton corresponding to the machine-harvested cotton canopy RGB image is suitable for harvesting, when the defoliation effect evaluation value is greater than the threshold value of the machine-harvested cotton defoliation effect comprehensive evaluation,
determining that the machine-harvested cotton corresponding to the machine-harvested cotton canopy RGB image is not suitable for harvesting when the defoliation effect evaluation value is less than or equal to the threshold value of the machine-harvested cotton defoliation effect comprehensive evaluation.

9. A method as claimed in claim 5-8 any item, wherein the comprehensive evaluation index of the machine-harvested defoliation effect comprises defoliation rate, boll-opening rate and yield.

10. A monitoring and evaluation system for comprehensive evaluation index of machine-harvested cotton defoliation effect, wherein comprises:
a machine-harvested cotton canopy RGB image acquisition module for acquiring machine-harvested cotton canopy RGB images;
an image feature extraction module for extracting visible-light vegetation index features, color component features and texture features of the machine-harvested cotton canopy RGB images;
a comprehensive evaluation model module for inputting the visible-light vegetation index features, color component features and texture features into a trained comprehensive evaluation model of machine-harvested cotton defoliation effect to output the defoliation effect evaluation values, the trained comprehensive evaluation model of machine-harvested cotton defoliation effect is an extreme learning machine model based on particle swarm optimization algorithm through training with visible-light vegetation index features, color component features and texture features of the machine-harvested cotton canopy RGB images as input and the defoliation effect evaluation value as output,
a machine-harvested cotton harvesting timing determination module for determining the harvesting timing of machine-harvested cotton based on the defoliation effect evaluation value.
